(19) United States
(12) Patent Application Publication
(10) Pub. No.: US 2005/0227294 A1
(43) Pub. Date: Oct. 13, 2005

(54) Molecular modification assays involving lipids

(75) Inventors: Elizabeth Gaudet, Menlo Park, CA (US); J. Richard Sportsman, Encinitas, CA (US)

Correspondence Address: KOLISCH HARTWELL, P.C., 520 S.W. YAMHILL STREET, SUITE 200, PORTLAND, OR, 97204, US

(73) Assignee: Molecular Devices Corporation

(21) Appl. No.: 11/146,553

(22) Filed: Jun. 6, 2005

Related U.S. Application Data
(63) Continuation-in-part of application No. 10/746,797, filed on Dec. 23, 2003.
Application No. 10/746,797 is a continuation-in-part of application No. 09/844,655, filed on Apr. 27, 2001.
Application No. 09/844,655 is a continuation-in-part of application No. PCT/US00/16025, filed on Jun. 9, 2000.
Application No. 09/844,655 is a continuation-in-part of application No. 09/596,444, filed on Jun. 19, 2000.
Application No. 09/596,444 is a continuation-in-part of application No. 08/929,095, filed on Sep. 15, 1997, now abandoned.
(60) Provisional application No. 60/577,079, filed on Jun. 4, 2004.
(60) Provisional application No. 60/602,712, filed on Aug. 18, 2004.
(60) Provisional application No. 60/615,308, filed on Sep. 30, 2004.
(60) Provisional application No. 60/436,725, filed on Dec. 26, 2002.
(60) Provisional application No. 60/507,006, filed on Sep. 29, 2003.
(60) Provisional application No. 60/241,032, filed on Oct. 17, 2000.
(60) Provisional application No. 60/223,642, filed on Aug. 8, 2000.
(60) Provisional application No. 60/200,594, filed on Apr. 28, 2000.

(51) Int. Cl. G01N 33/53

(52) U.S. Cl. 435/7.1

(57) ABSTRACT

Assays for detecting molecular modifications, such as phosphorylation, dephosphorylation, and/or cleavage, among others, of lipids, lipid fragments, and/or lipid precursors. These assays may be used to detect the presence and/or activity of enzymes and/or other agents, such as drugs, involved in facilitating, inhibiting, and/or otherwise regulating such modifications.

### CROSS-REFERENCE TO PRIORITY APPLICATIONS

This application is a continuation-in-part of U.S. patent application Ser. No. 10/746,797, filed Dec. 23, 2004. This application also is based upon and claims the benefit under 35 U.S.C. § 119(e) of the following U.S. provisional patent applications: Ser. No. 60/577,079, filed Jun. 4, 2004; Ser. No. 60/602,712, filed Aug. 18, 2004; and Ser. No. 60/615,308, filed Sep. 30, 2004.

U.S. patent application Ser. No. 10/746,797, in turn, is a continuation-in-part of U.S. patent application Ser. No. 09/844,655, filed Apr. 27, 2001. U.S. patent application Ser. No. 10/746,797 also is based upon and claims the benefit under 35 U.S.C. § 119(e) of the following U.S. provisional patent applications: Ser. No. 60/436,725, filed Dec. 26, 2002; and Ser. No. 60/507,006, filed Sep. 29, 2003.

U.S. patent application Ser. No. 09/844,655, in turn, is a continuation-in-part of the following patent applications: PCT Patent Application Serial No. PCT/US00/16025, filed Jun. 9, 2000; and U.S. patent application Ser. No. 09/596,444, filed Jun. 19, 2000. U.S. patent application Ser. No. 09/844,655 also is based upon and claims the benefit under 35 U.S.C. § 119(e) of the following U.S. provisional patent applications: Ser. No. 60/200,594, filed Apr. 28, 2000; Ser. No. 60/223,642, filed Aug. 8, 2000; and Ser. No. 60/241,032, filed Oct. 17, 2000.

PCT Patent Application Serial No. PCT/US00/16025, in turn, is a continuation-in-part of U.S. patent application Ser. No. 09/349,733, filed Jul. 8, 1999. PCT Patent Application Serial No. PCT/US00/16025 also is based upon and claims the benefit under 35 U.S.C. § 119(e) of the following U.S. provisional patent applications: Ser. No. 60/138,311, filed Jun. 9, 1999; Ser. No. 60/138,438, filed Jun. 10, 1999; and Ser. No. 60/200,594, filed Apr. 28, 2000.

U.S. patent application Ser. No. 09/596,444, in turn, is a continuation-in-part of the following patent applications: U.S. patent application Ser. No. 08/929,095, filed Sep. 15, 1997; and PCT Patent Application Serial No. PCT/US00/16025, with priority claims as indicated above.

U.S. patent application Ser. No. 09/349,733, in turn, claims priority from U.S. Provisional Patent Application Ser. No. 60/092,203, filed Jul. 9, 1998.

Each of the above-identified U.S., PCT, and provisional patent applications is incorporated herein by reference in its entirety for all purposes.

### CROSS-REFERENCES TO OTHER MATERIALS

This application incorporates by reference the following U.S. Pat. No. 5,843,378, issued Dec. 1, 1998; U.S. Pat. No. 6,965,381, issued Oct. 12, 1999; U.S. Pat. No. 6,071,748, issued Jun. 6, 2000; and U.S. Pat. No. 6,097,025, issued Aug. 1, 2000.

This application also incorporates by reference the following U.S. patent applications: Ser. No. 08/840,553, filed Apr. 14, 1997; U.S. Ser. No. 09/118,141, filed Jul. 16, 1998; U.S. Ser. No. 09/144,578, filed Aug. 31, 1998; U.S. Ser. No. 09/156,318, filed Sep. 18, 1998; U.S. Ser. No. 09/478,819, filed Jan. 5, 2000; U.S. Ser. No. 09/626,208, filed Jul. 26, 2000; U.S. Ser. No. 09/643,221, filed Aug. 18, 2000; U.S. Ser. No. 09/710,061, filed Nov. 10, 2000; U.S. Ser. No. 09/722,247, filed Nov. 24, 2000; U.S. Ser. No. 09/733,370, filed Dec. 8, 2000; U.S. Ser. No. 09/759,711, filed Jan. 12, 2001; U.S. Ser. No. 09/765,869, filed Jan. 19, 2001; U.S. Ser. No. 09/765,874, filed Jan. 19, 2001; U.S. Ser. No. 09/766,131, filed Jan. 19, 2001; U.S. Ser. No. 09/767,316, filed Jan. 22, 2001; U.S. Ser. No. 09/767,434, filed Jan. 22, 2001; U.S. Ser. No. 09/767,579, filed Jan. 22, 2001; U.S. Ser. No. 09/767,583, filed Jan. 22, 2001; U.S. Ser. No. 09/768,661, filed Jan. 23, 2001; U.S. Ser. No. 09/768,742, filed Jan. 23, 2001; U.S. Ser. No. 09/768,765, filed Jan. 23, 2001; U.S. Ser. No. 09/770,720, filed Jan. 25, 2001; U.S. Ser. No. 09/770,724, filed Jan. 25, 2001; U.S. Ser. No. 09/777,343, filed Feb. 5, 2001; U.S. Ser. No. 09/813,107, filed Mar. 19, 2001; U.S. Ser. No. 09/815,932, filed Mar. 23, 2001; and U.S. Ser. No. 09/836,575, filed Apr. 16, 2001.

This application also incorporates by reference the following PCT patent applications: Serial No. PCT/US98/23095, filed Oct. 30, 1998; Serial No. PCT/US99/01656, filed Jan. 25, 1999; Serial No. PCT/US99/03678, filed Feb. 19,1999; and Serial No. PCT/US99/08410, filed Apr. 16, 1999.

This application also incorporates by reference the following U.S. provisional patent applications: Ser. No. 60/092,203, filed Jul. 9, 1998; Ser. No. 60/094,275, filed Jul. 27, 1998; Ser. No. 60/094,276, filed Jul. 27, 1998; Ser. No. 60/094,306, filed Jul. 27, 1998; Ser. No. 60/100,817, filed Sep. 18, 1998; Ser. No. 60/100,951, filed Sep. 18, 1998; Ser. No. 60/104,964, filed Oct. 20,1998; Ser. No. 60/114,209, filed Dec. 29, 1998; Ser. No. 60/116,113, filed Jan. 15, 1999; Ser. No. 60/117,278, filed Jan. 26, 1999; Ser. No. 60/119,884, filed Feb. 12, 1999; Ser. No. 60/121,229, filed Feb. 23, 1999; Ser. No. 60/124,686, filed Mar. 16, 1999; Ser. No. 60/125,346, filed Mar. 19, 1999; Ser. No. 60/126,661, filed Mar. 29, 1999; Ser. No. 60/130,149, filed Apr. 20, 1999; Ser. No. 60/132,262, filed May 3, 1999; Ser. No. 60/132,263, filed May 3, 1999; Ser. No. 60/135,284, filed May 21, 1999; Ser. No. 60/136,566, filed May 28, 1999; Ser. No. 60/138,737, filed Jun. 11, 1999; Ser. No. 60/138,893, filed Jun. 11, 1999; and Ser. No. 60/142,721, filed Jul. 7, 1999; Ser. No. 60/178,026, filed Jan. 26, 2000; Ser. No. 60/222,222, filed Aug. 1, 2000; Ser. No. 60/244,012, filed Oct. 27, 2000; Ser. No. 60/250,681, filed Nov. 30, 2000; Ser. No. 60/250,683, filed Nov. 30, 2000; Ser. No. 60/267,639, filed Feb. 10, 2001; and Ser. No. 60/369,704, filed Apr. 2, 2002.

This application also incorporates by reference the following publications: Richard P. Haugland, Handbook of Fluorescent Probes and Research Chemicals (6^{th} ed. 1996); Joseph R. Lakowicz, Principles of Fluorescence SDectroscopy (2^{nd} Edition 1999); and Bob Sinclair, Everything's Great When It Sits on a Chip: A Bright Future for DNA Arrays, 13 THE SCIENTIST, May 24,1999, at 18.

### INTRODUCTION

The physiological modification of molecules and supramolecular assemblies plays a major role in the structure and regulation of biological systems. These modifications may include “local” modifications, such as phosphorylation, prenylation, cyclization, glycosylation, carboxylation, acylation, and/or sulfonation of amino acids, nucleotides, and/or lipids, among others. These modifications also or alternatively may include “global” modifications, such as cleavage or ligation of proteins, nucleic acids, and/or lipids. In these global modifications, one molecule may be split into two or more molecules (or fragments), or two or more molecules may be joined to form one molecule. The prevalence and significance of molecular modifications make it particularly likely that errors in modifications and/or errors in the regulation of such modifications will lead to disease and/or other pathologies. Thus, there is intense interest both in characterizing molecular modifications and in understanding their regulation. There is also intense interest in identifying activating and/or inhibitory drugs to modulate molecular modifications.

Lipid modifications play an important role in cellular signaling. In particular, lipid modifications may affect signal transduction at membranes through various signaling pathways, to affect cell motility, proliferation, survival, membrane trafficking, and/or carbohydrate metabolism, among others.

Phosphorylation/dephosphorylation of lipids at cellular membranes may contribute to the recruitment and/or release of various signaling components, to activate or inhibit the signaling components. For example, a phosphoinositide kinase, PI3-kinase (PI3K), may phosphorylate phosphatidylinositol 4,5-bisphosphate (PIP2) at the membrane to form phosphatidylinositol 3,4,5-triphosphate (PIP3). PIP3 may recruit the kinases PKB/Akt and PDK1 to the membrane, where PDK1 may phosphorylate PKB/Akt. This phosphorylated PKB/Akt then may translocate to the nucleus to phosphorylate nuclear proteins and thus regulate nuclear events, such as gene transcription. Phosphoinositide phosphatases (such as MTM1 and PTEN) may remove the phosphate added by PI3-kinase, to release PKB/Akt and PDK1 from the membrane and thereby attenuate the action of PKB/Akt. These phosphoinositide kinases and phosphatases thus may exert counterbalancing effects on cell regulation. For example, PI3-kinase may promote growth, motility, and/or cancer, and PTEN and/or MTM1 may function to restrict these cell activities.

In some cases, lipids may transduce signals in cells by modifications that cleave the lipids into lipid fragments. For example, phospholipase C cleaves PIP2, to produce the second messengers inositol triphosphate (IP3) and diacylglycerol. The inositol triphosphate is water soluble and diffuses to the endoplasmic reticulum (ER), where it releases Ca²⁺ from the ER by binding to IP3-gated Ca²⁺-release channels in the ER membrane. The diacylglycerol is membrane bound and may be cleaved to form the second messenger arachidonic acid or may activate the Ca²⁺-dependent serine/threonine kinase protein kinase C that in turn activates or inhibits other enzymes to effect a response.

Increasing interest in lipid signaling has expanded the demand for assays that measure lipid modification by enzymes. Traditional assays may employ radioactivity, for example, by applying a radioactive tag to a lipid. The tagged lipid may be exposed to an enzyme and then fractionated by low-throughput techniques, such as thin-layer chromatography or high-performance liquid chromatography, to detect modification of the lipid. Because they involve radioactivity, these assays present a short-term safety hazard for assay operators and a long-term storage and disposal problem. Furthermore, these low-throughput techniques for fractionation may not be suitable for high-throughput assays.

### SUMMARY

The present teachings provide assays for detecting molecular modifications, such as phosphorylation, dephosphorylation, and/or cleavage, among others, of lipids, lipid fragments, and/or lipid precursors. These assays may be used to detect the presence and/or activity of enzymes and/or other agents, such as drugs, involved in facilitating, inhibiting, and/or otherwise regulating such modifications.

### BRIEF DESCRIPTION OF THE DRAWINGS

FIG. 1 is a schematic view of species and/or reactions that may be analyzed using molecular modification assays, in accordance with aspects of the present teachings.

FIG. 2 is a flowchart showing steps that may be used alone, together, and/or in combination with other steps to construct molecular modification assays, in accordance with aspects of the present teachings.

FIG. 3 is a schematic view of exemplary lipid modification assays involving lipid phosphorylation and/or dephosphorylation, in accordance with aspects of the present teachings.

FIG. 4 is a schematic view of exemplary lipid modification assays involving lipid cleavage and/or ligation, in accordance with aspects of the present teachings.

FIG. 5 is a schematic view of exemplary lipid modification assays involving lipid cleavage and/or lipid phosphorylation/dephosphorylation and performed as a competition using a luminescent target, in accordance with aspects of the present teachings.

FIG. 6 is a schematic view of an exemplary assay for phosphoinositide phosphatase activity, in accordance with the present teachings.

FIG. 7 is a plot of fluorescence polarization measured as a function of MTM1 enzyme concentrations in assays performed generally as shown in FIG. 6, in accordance with aspects of the present teachings.

### DETAILED DESCRIPTION

The present teachings provide assays for detecting molecular modifications, such as phosphorylation, dephosphorylation, and/or cleavage, among others, of lipids, lipid fragments, and/or lipid precursors. These assays may be used to detect the presence and/or activity of enzymes and/or other agents, such as drugs, involved in facilitating, inhibiting, or otherwise regulating such lipid modifications.

The molecular modifications may include structural changes in lipids, lipid fragments, and/or lipid precursors, such as phosphate addition and/or removal, among others. Alternatively, or in addition, the molecular modifications may include cleavage of a lipid into lipid fragments, such as by a lipase enzyme, and/or joining of two lipid precursors, such as by a lipid ligase enzyme, among others. The present teachings may include a method of detecting modification of a lipid (and/or lipid fragment and/or lipid precursor) in a sample, where the method includes contacting the sample with a binding partner that binds specifically to one of the modified and nonmodified forms of the lipid (and/or fragment and/or precursor), but not both, the binding partner including a metal required for specific binding to the one form; and detecting a response indicative of the extent of binding between the binding partner and the one form.

The assays may include luminescence assays, such as luminescence polarization, luminescence resonance energy transfer, and/or luminescence intensity, among others. The assays provided by the present teachings may be useful in a variety of applications, including, without limitation, life science research, drug research, accelerated drug discovery, assay development, and high-throughput screening. Further aspects of the invention are described in the following sections, including, among others, (I) definitions, (II) overview, (III) assays, and (IV) examples.

### I. DEFINITIONS

The various technical terms used herein generally have the meanings that are commonly recognized by those skilled in the art. However, the following terms may have additional and/or alternative meanings, as described below:

Luminescent—capable of, suitable for, or exhibiting luminescence, which is the emission of light by sources other than a hot, incandescent body. Luminescence may be caused by electronic transitions within a luminescent substance (or luminophore) from more energetic to less energetic states. Among several types are chemiluminescence, electrochemiluminescence, electroluminescence, photoluminescence, and triboluminescence, which are produced by chemical reactions, electrochemical reactions, electric discharges, absorption of light, and the rubbing or crushing of crystals, respectively. Molecules may be intrinsically and/or extrinsically luminescent, meaning that they are luminescent on their own or luminescent due to covalent and/or noncovalent association with another molecule that is luminescent. Exemplary luminescent molecules and mechanisms for producing luminescent molecules are described in U.S. patent application Ser. No. 09/815,932, filed Mar. 23, 2001; and in Richard P. Haugland, Handbook of Fluorescent Probes and Research Products (9^{th} ed. 2002), each of which is incorporated herein by reference.

Lipid—a hydrophobic compound soluble in various organic solvents. Lipids may include fats, oils, waxes, and/or the like. Lipids may be hydrophobic throughout, or may include polar moieties, such as a polar head group. Lipids may be water insoluble or water soluble.

Lipids may include one or more hydrocarbon chains of any suitable length. Exemplary hydrocarbon chains may include about 4-30 carbon atoms, and may be unbranched or branched, and noncylic or cyclic. Each chain may be saturated or unsaturated. Unsaturated hydrocarbon chains may include any suitable number of double (or triple) bonds, including one, two, three, or more. The hydrocarbon chains may be included in fatty acids (or fatty acid derivatives, such as esters or amides, among others). Exemplary fatty acids are listed below in Table 1:

| Number of | | |
|---|---|---|
| Carbons | Saturated Fatty Acid | Unsaturated Fatty Acid |
| 4 | Butyric acid | Crotonic acid |
| 6 | Caproic acid | Palmitoleic acid |
| 8 | Caprylic acid | Oleic acid |
| 10 | Capric acid | Linoleic acid |
| 12 | Lauric acid | Linolenic acid |
| 14 | Myristic acid | Arachidonic acid |
| 16 | Palmitic acid | Nervonic acid |
| 18 | Stearic acid | |
| 20 | Arachidic acid | |
| 24 | Lignoceric acid | |

In some examples, the lipids may be derivatives of glycerol or sphingosine. Exemplary glycerol derivatives may include one, two, or three fatty acid chains covalently linked to the glycerol (mono-, di-, and triglycerides, respectively). In some examples, the lipids may include two fatty acids chains, one that is saturated and another that is unsaturated. Exemplary sphingosine derivatives may include sphingosine 1-phosphate, ceramide, and/or glycosphingolipids such as cerebrosides, sulfatides, globosides, and/or gangliosides.

In some examples, the lipids may include cholesterol or derivatives thereof. Accordingly, the lipids may be steroids or steroid-like compounds. Exemplary steroids may include glucocorticoids (cortisol, cortisone, hydrocortisone, etc.), mineralocorticoids, vitamin D, progesterones, estrogens (β-estradiol, estriol, estrone, etc.), androgens (testosterone, dihydrotestosterone, etc.), and/or the like.

In some examples, the lipids may be ecosanoids. Exemplary ecosanoids may include prostaglandins (PGs, such as PGE₂), thromboxanes (TXs, such as TXA₂), and/or leukotrienes (LTs, such as LTA₄).

The lipids may be phospholipids including one, two, three, or more phosphate groups covalently linked to the lipids and/or to each other. The phosphate groups may be disposed at an end of the lipid, that is, primary phosphate groups linked to the lipid by a single phosphoester bond. Alternatively, or in addition, the phosphate groups may be disposed more centrally in the lipids, that is, secondary phosphate groups (generally phosphodiesters), linked to the lipid by two or more covalent bonds, such as phosphatidylinositol, cyclic phosphodiesters, and/or derivatives thereof. In some examples, phosphodiesters (cyclic or noncyclic secondary phosphates) may not bind substantially to a binding partner until they are hydrolyzed to mono-phosphoesters (primary phosphates).

In some examples, the lipids may be glycolipids including at least one carbohydrate moiety. The carbohydrate moiety may be included in a polar head group and may have any suitable number of sugar units, such as a monosaccharide, a disaccharide, a trisaccharide, etc. Exemplary carbohydrates may include inositol, glucose, galactose, N-acetyl galactose (GaINAc), and/or the like. Exemplary glycolipids may include phosphatidylinositol and its derivatives, and/or glycosphingolipids (such as cerebrosides, sulfatides, globosides and/or gangliosides), among others.

The lipids may include any suitable polar head groups. The polar head groups may include sugars, such as those described above, glycerol, amines, amino acids or derivatives thereof, peptides or proteins, phosphate, and/or the like. Accordingly, exemplary polar head groups may include phosphoinositiol, phosphoethanolamine, phosphoserine, phosphothreonine, phosphocholine, phosphotaurine, phosphoglycerol, nonphosphorylated derivatives thereof, etc.

Lipid fragments—molecules produced by cleavage of lipids, such as diacylglycerols, sugars, phosphosugars, phosphoamino acids, and/or derivatives thereof.

Lipid precursors—molecules that correspond to structural portions of lipid, such as sugars, phosphosugars, amino acids, phosphoamino acids, fatty acids, diacylgylcerols, and/or derivatives thereof.

Specific binding—binding to a specific binding partner to the exclusion of binding to most other moieties. Specific binding can be characterized by a dissociation constant or coefficient (alternatively termed an affinity or binding constant or coefficient). Generally, dissociation constants for specific binding range from 10⁻⁴ M to 10⁻¹² M and lower, and preferred dissociation constants for specific binding range from 10⁻⁸ or 10⁻⁹ M to 10⁻¹² M and lower.

Lipase—any enzyme that catalyzes the splitting or degradation of lipids into smaller lipid components or fragments, such as fatty acids, diacylglycerols, polar head groups (such as sugars or phosphosugars), etc. Such degradation may be targeted to specific lipid types or lipid regions. For example, the lipase may cleave an ester linkage of a fatty acid on the first or second carbon of a glycerol backbone (such as with phospholipase A₁ or A₂, respectively), or may cleave a phosphate bond of a lipid (such as with phospholipase C or D), among others.

Lipid Ligase—an enzyme that catalyzes creation of a covalent bond between lipid precursors resulting in formation of a lipid.

### II. OVERVIEW

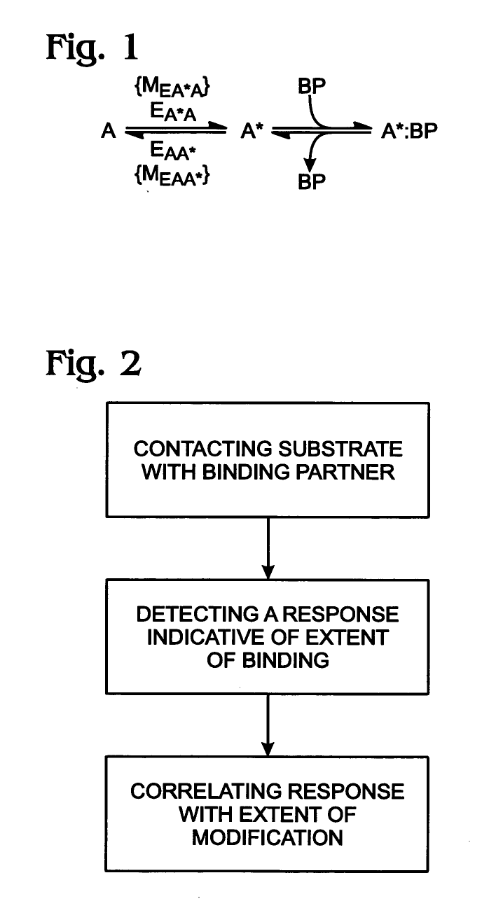

FIG. 1 shows species and/or reactions that may be analyzed using assays provided by the present teachings. The species include reactant and product A and A*, respectively, enzymes E_{AA}* and E_{A}*_{A}, and/or enzyme modulators M_{EAA}* and M_{E}*_{A}, among others. The assays may be used to analyze the presence and/or quantity of a binding target, A and/or A*. Alternatively, or in addition, the assays may be used to analyze the presence, quantity, and/or activity of E_{AA}*, E_{A}*_{A}, M_{EAA}*, and/or M_{EA}*_{A}. Quantity refers generally to amount, which may be defined intrinsically and/or extrinsically, for example, using concentration, and/or number or mass, respectively. Activity refers generally to rate, which may be defined as the rate of substrate consumption and/or product formation per time. Here, quantity and/or amount may be used so as to encompass the simple presence of components, and activity and/or rate may be used so as to encompass the simple presence of activity.

Binding targets A and A* generally comprise any two lipid species related by a modification (denoted by the presence or absence of *). A and A* may include lipid molecules, lipid fragments, and/or lipid precursors, and/or assemblies of such molecules, fragments, and/or precursors. The modification may include addition, subtraction, oxidation, reduction, cyclization, decyclization, ligation, and/or cleavage modifications, among others. A and A* may be related as substrate and product in a reaction, such as an enzyme-catalyzed reaction. In some embodiments, A and/or A* may include components intended to facilitate detection of binding between A or A* and BP, such as a luminophore, a quencher, an energy transfer partner, a selected functional group, and the like.

BP generally comprises any binding partner capable of binding specifically to binding target A or A* (i.e., the modified species or the unmodified species) but not to both. BP may include any binding partner having the specified binding properties. In some examples, BP may not include a polypeptide and/or an immunoglobulin, and/or a functional portion or fragment thereof.

In other examples, BP may include a polypeptide or a peptide that binds to a lipid, lipid precursor, or lipid fragment. For example, proteins that specifically bind phosphoinositides may be used as binding partners, such as FYVE-containing proteins and/or proteins with PH domains. As another example, PHISH may be included to bind Ptdins(3,4,5)P₃. Other proteins that bind to inositol phosphates may be used as binding partners, for example, centaurina binds specifically to inositol 4-phosphate. A protein or a peptide including one of these binding moieties may be used to measure the activity of phosphoinositides and/or inositol pathway enzymes. For example, in a PI3 kinase (PI3K) assay, PI3K in the presence of ATP phosphorylates fluorescent Ptdins(4,5)P₂ substrate to make Ptdins(3,4,5)P₃. The 3-phosphorylated product then may be detected in fluorescence polarization assay, such as with the PHISH protein, which binds specifically to the Ptdlns(3,4,5)P₃ product of the reaction, as binding partner. Binding partners with polypeptides or peptides may include or lack a metal.

BP may include a metal. In some examples, the metal may be a metal ion. The metal may be configured to form a metal-ligand coordination complex with the binding target, in which one or more electrons are shared between the metal and the binding target. For example, the metal may be a Lewis acid and particularly a strong Lewis acid. In exemplary embodiments, the metal is a Lewis acid that shares electrons with a Lewis base, such as a phosphate. Accordingly, binding between BP and the binding target may be a covalent interaction and/or may be a charge-charge interaction, among others. Exemplary metals that may be suitable as part (or all) of the binding partner include aluminum, chromium, europium, gallium, iridium, iron, manganese, osmium, platinum, rhenium, ruthenium, scandium, strontium, terbium, titanium, vanadium, yttrium, and/or zirconium, among others. In some examples, the metals may be aluminum, gallium, and/or iron. Alternatively, or in addition, the metals may be strontium, europium, terbium, and/or zirconium.

The metal may be in ionic form. Accordingly, BP may be, include, and/or be formed from a metal salt. BP may include one or more metal ions, including dicationic, tricationic, tetracationic, and/or other polycationic metal ions, among others. Suitable dicationic metal ions may include europium (Eu²⁺), iridium (Ir²⁺), osmium (Os²⁺), platinum (Pt²⁺), rhenium (Re²⁺), ruthenium (Ru²⁺), and/or strontium (Sr²⁺), among others. Suitable tricationic metal ions may include aluminum (Al³⁺), chromium (Cr³⁺), europium (Eu³⁺), gallium (Ga³⁺), iron (Fe³⁺), manganese (Mn³⁺), scandium (Sc³⁺), terbium (Tb⁺³), titanium (Ti³⁺), vanadium (V³⁺), and/or yttrium (Y³⁺), among others. Suitable tetracationic metal ions may include zirconium (Zr⁴⁺).

A metal or metal ion may be a binding species of BP required for interaction with binding target A or A*. BP may be selected to interact with one or more functional groups present on one of A or A*. The functional groups may be the result of modification, for example, the phosphate group on a phosphorylated lipid. In these examples, BP may bind to a substrate such as A or A* only if it is phosphorylated, where the binding between the substrate and the binding partner is substantially nonspecific with respect to the structure of the substrate aside from any phosphate groups. Thus, the binding may occur substantially without regard to the particular lipid structure. In some examples, BP may be selected to bind to A or A* only if A or A* possesses an appropriate functional group. The functional group may be selected based upon its ionic properties, and may include, for example, functional groups that are ionic at physiological pH levels, and thereby bind to the selected BP. Selected functional groups that may be appropriate for selection to bind with BP include phosphate, carboxylate, and/or sulfonate, among others.

In some examples, BP may include a distinct structure to which the metal is connected or otherwise associated. The distinct structure may be a macromolecule (a protein, nucleic acid, polysaccharide, polymer, and/or the like) and/or an associated solid support (or solid phase). The solid support may be a particle, a membrane, and/or a sample holder (such as a microplate), among others. Here, particles include nanoparticles, microparticles, and macroparticles, among others, where nanoparticles are particles dimensions less than about 100 nm, microparticles are particles with dimensions between about 100 nm and about 10 μm, and macroparticles are particles with dimensions between about 10 μm and about 1 mm. The metal may be associated with (or “tethered”) to the macromolecule and/or solid support using any suitable linkage mechanism, including hydrogen bonding, ionic bonding, electrostatic binding, hydrophobic interactions, Van der Waals interactions, and/or covalent attachment, among others. Alternatively, or in addition, the metal may be “untethered,” that is, not connected to a distinct structure substantially lacking the metal. However, an untethered metal may be included in a metal-based complex, particularly a macromolecular complex, such as an aggregate, microcrystal, and/or a metal hydroxide/oxide, among others. In some embodiments, the complex may form from a metal (or metal salt) when the metal (or metal salt) is placed in a suitable aqueous environment. The suitable aqueous environment may include buffer components (including pH-modifying components such as an acid or base). In some embodiments, BP may include components intended to facilitate detection of binding between BP and A or A*, such as a luminophore, a quencher, an energy transfer partner, and the like.

Metal salts forming a metal-based complex included in the binding partner may have any suitable formula weight. As used herein, the formula weight is the mass of the smallest repeating unit of the metal salt. The formula weight generally is defined with the metal salt in an anhydrous or hydrated crystalline form, that is, before placed in a liquid. With this definition, water-mediated reactions that produce bridged compounds, hydroxides/oxides, aggregates, etc. are ignored in calculating the formula weight. For example, the metal salt gallium chloride may form larger complexes in certain aqueous environments, but has a formula weight (anhydrous) of about 176 grams/mole. In some embodiments, the metal salt may have a formula weight of less than about 10⁵, 10⁴, 10³, or 400 grams/mole. Alternatively, or in addition, the metal salt may have a formula weight that is less than the formula or molecular weight of the binding target (for example, the substrate or product), or less than about ten-fold or one-hundred fold the formula or molecular weight of the binding target.

E_{AA}* and E_{A}*_{A} generally comprise any enzymes or other catalysts capable of facilitating reactions converting A to A* and A* to A, respectively. E_{AA}* and E_{A}*_{A} may include, among others, enzymes such as kinases, phosphatases, and/or lipases, among others.

Selected lipid-modifying enzymes and corresponding substrates and inhibitors are provided in the following table:

TABLE 1
Exemplary Lipid-Modifying Enzymes, Substrates, and Inibitors

| Enzyme | Exemplary Substrate(s) | Selected Inhibitors |
|---|---|---|
| Lipid Kinases | | |
| PI3-Kinases | Phosphatidylinositol | Wortmannin; |
| | (Ptd(Ins)) | LY294002 |
| Spingosine Kinases | Sphingosine | N,N-dime- |
| | | thylsphingosine |
| Lipid Kinases | | |
| Phospholipase A1 | Ptd(Ins)4,5P₂ | |
| Phospholipase A2 | Ptd(Ins)4,5P₂ | |
| Phospholipase C | Ptd(Ins)4,5P₂ | U73122 |
| Phospholipase D | Ptd(Ins)4,5P₂ | |
| Lipid Phosphatases | | |
| PTEN | Ptd(Ins)3P | |
| MTM1/Myotubularin | Ptd(Ins)3P | |
| Inositol Phosphatases | | |
| Inositol | Inositol 1-phosphate | Lithium |
| monophosphatase | | |

M_{EAA}* and M_{EA}*_{A} generally comprise any modulators or other agents capable of modulating or otherwise affecting the activity of E_{AA}* and E_{A}*_{A}, respectively. The modulator may be a change in environmental condition, such as a change in sample temperature, but more typically is an enzyme or other reagent added to the sample. The modulator may be a chemical reagent, such as an acid, base, metal ion, organic solvent, and/or other substance intended to effect a chemical change in the sample. Alternatively, or in addition, the modulator may have or be suspected to have a biological activity or type of interaction with a given biomolecule, such as an enzyme, a small compound (a drug candidate), oligonucleotide, nucleic acid polymer, peptide, protein, and/or other biologically active molecule. The modulator may include an agonist or inhibitor capable of promoting or inhibiting, respectively, the activity of the modulated enzyme.

Further aspects of exemplary lipid modification assays are included below in Section IV.

### III. ASSAYS

FIG. 2 shows several steps that may be used alone, together, and/or in combination with other steps to construct assays according to various aspects of the present teachings. These steps may include (a) contacting at least one member of a pair of molecules or other entities related by a modification, as described herein, with a binding partner capable of binding one of the pair of molecules but not the other, as described herein, (b) detecting a response indicative of the extent of binding between the at least one member of the pair and the binding partner, and (c) correlating the response with (i) the kinetics and/or extent of the modification, and/or (ii) with the presence and/or activity of an enzyme that affects the modification. The assays further may include contacting the at least one member with the enzyme before and/or after the steps of contacting, detecting, and correlating. The assays further may include contacting the at least one member and the enzyme with a candidate compound such as a putative modulator before and/or after the step of contacting the at least one member with the enzyme, and determining the ability of the candidate compound to promote or inhibit the modification by its effects on the kinetics and/or extent of binding. Alternatively, or in addition, the assays further may include washing the sample including the at least one member and the binding partner to remove any member of the pair not bound to the binding partner prior to the step of detecting the extent of binding. In some embodiments, the assays may include repeating the steps of contacting, detecting, and/or correlating for the same sample and/or a plurality of different samples. For example, the assays may involve providing a sample holder having a plurality of sample sites supporting a corresponding plurality of samples, and sequentially and/or simultaneously repeating the steps of contacting, detecting, and/or correlating, among others, for the plurality of samples. The remainder of this section describes in more detail the steps of (a) contacting, (b) detecting, and (c) correlating. The description of these steps is augmented by the disclosure in the following patent applications, which are incorporated herein by reference: U.S. patent application Ser. No. 09/844,655, filed Apr. 27, 2001; and U.S. Provisional Patent Application Ser. No. 60/507,006, filed Sep. 29, 2003.

A. Contacting

The step of contacting assay components such as enzymes, enzyme modulators, substrates, products, and/or binding partners with one another and/or with other species generally comprises any method for bringing any specified combination of these components into functional and/or reactive contact. A preferred method is by mixing and/or forming the materials in solution, although other methods, such as attaching one or more components such as the binding partner to a bead or surface, also may be used, as long as the components retain at least some function, specificity, and/or binding affinity following such attachment. Exemplary apparatus having fluidics capability suitable for contacting or otherwise preparing assay components are described in the following patent applications, which are incorporated herein by reference: U.S. patent application Ser. No. 09/777,343, filed Feb. 5, 2001; and U.S. patent application Ser. No. 10/061,416, filed Feb. 1, 2002.

One or more of the assay components may comprise a sample, which typically takes the form of a solution containing one or more biomolecules that are biological and/or synthetic in origin. The sample may be a biological sample that is prepared from a blood sample, a urine sample, a swipe, or a smear, among others. Alternatively, the sample may be an environmental sample that is prepared from an air sample, a water sample, or a soil sample, among others. The sample typically is aqueous but may contain biologically compatible organic solvents, buffering agents, inorganic salts, and/or other components known in the art for assay solutions.

Contacting may be performed in the presence or absence of liposomes (lipid vesicles), micelles, and/or lipid solubilizing agents, among others. Exemplary liposomes may be produced from biological membranes, such as disrupted and/or fractionated cells and/or may be prepared artificially, such as from fractionated lipids. Exemplary micelles (or liposomes) may include ionic or nonionic detergents, such as TritonX-100, CHAPS, Brij, etc. Lipid solubilizing agents may include an effective amount of any substance(s) that places lipid substrates/products in a suitably soluble condition to perform modification assays. Exemplary solubilizing agents may include detergents, organic solvents, and/or the like. In some examples, contacting may include a step of altering liposomes and/or micelles, such as disrupting or changing the size of these structures, by addition of salt, an organic solvent (such as ethanol), and/or the like. The step of altering may be performed at any suitable time, including before, during, and/or after an enzyme reaction and/or a step of contacting with a binding partner.

The assay components and/or sample(s) may be supported for contact and/or analysis by any substrate or material capable of and/or configured or adapted for providing such support. Suitable substrates include microplates, biochips, cuvettes, and test tubes, among others. Here, features such as microplate wells and microarray (i.e., biochip) sites may comprise assay sites. Exemplary microplates are described in U.S. Pat. No. 6,488,892, issued Dec. 3, 2002, which is incorporated herein by reference. These microplates may include 96, 384, 1536, and/or other numbers of wells. The wells, in turn, optionally may have small (≦50 μL) volumes, elevated bottoms, and/or frusto-conical shapes capable of matching a sensed volume, among others.

B. Detecting

The step of detecting a response indicative of the kinetics and/or extent of binding generally comprises any method for effectuating such detection, including detecting and/or quantifying a change in, or an occurrence of, a suitable parameter and/or signal. The method may include luminescence and/or nonluminescence methods, and heterogeneous and/or homogeneous methods, among others.

Luminescence and nonluminescence methods may be distinguished by whether they involve detection of light emitted by a component of the sample. Luminescence assays involve detecting light emitted by a luminescent compound (or luminophore) and using properties of that light to understand properties of the compound and its environment. A typical luminescence assay may involve (1) exposing a sample to a condition capable of inducing luminescence from the sample, and (2) measuring a detectable luminescence response, for example, indicative of the extent of binding or other interaction between the member of interest and a corresponding binding partner. Most luminescence assays are based on photoluminescence, which is luminescence emitted in response to absorption of suitable excitation light. However, luminescence assays also may be based on chemiluminescence, which is luminescence emitted in response to chemical excitation, and electrochemiluminescence, which is luminescence emitted in response to electrochemical energy. Suitable luminescence assays include, among others, (1) luminescence intensity, which involves detection of the intensity of luminescence, (2) luminescence polarization, which involves detection of the polarization of light emitted in response to excitation by polarized light, and (3) luminescence energy transfer, which involves detection of energy transfer between a luminescent donor and a suitable acceptor. Nonluminescence assays involve using a detectable response other than light emitted by the sample, such as absorption, scattering, and/or radioactivity, among others. Exemplary luminescence and nonluminescence assays, and information and procedures pertaining thereto, are described in the following materials, which are incorporated herein by reference: U.S. Pat. No. 6,466,316, issued Oct. 15, 2002; and Joseph R. Lakowicz, Principles of Fluorescence Spectroscopy (2^{nd} ed. 1999).

The detectable luminescence response, in the case of luminescence assays, generally comprises a change in, or an occurrence of, a luminescence signal that is detectable by direct visual observation and/or by suitable instrumentation. Typically, the detectable response is a change in a property of the luminescence, such as a change in the intensity, polarization, energy transfer, lifetime, and/or excitation or emission wavelength distribution of the luminescence. The detectable response may be simply detected, and/or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence assays, the detectable response may be generated directly using a luminophore associated with an assay component actually involved in binding such as A* or BP, or indirectly using a luminophore associated with another (e.g., reporter or indicator) component. Suitable luminophores and methods for luminescently labeling assay components are described in Richard P. Haugland, Handbook of Fluorescent Probes and Research Products (9^{th} ed. 2002), which is incorporated herein by reference.

Heterogeneous and homogeneous methods may be distinguished by whether they involve sample separation before detection. Heterogeneous methods generally require bulk separation of bound and unbound species. This separation may be accomplished, for example, by washing away any unbound species following capture of the bound species on a solid phase, such as a bead or microplate surface labeled with a trivalent metal ion or other suitable binding partner. The extent of binding then can be determined directly by measuring the amount of captured bound species and/or indirectly by measuring the amount of uncaptured unbound species (if the total amount is known). Homogeneous methods, in contrast, generally do not require bulk separation but instead require a detectable response such as a luminescence response that is affected in some way by binding or unbinding of bound and unbound species without separating the bound and unbound species. For example, enzyme activity may result in covalent or high affinity binding of relatively small luminescent molecules to relatively large molecules, with a concomitant decrease in the small molecules' mobility (e.g., as detectable using luminescence polarization). Homogeneous assays typically are simpler to perform but more complicated to develop than heterogeneous assays.

C. Correlating

The step of correlating generally comprises any method for correlating or relating the kinetics and/or extent of binding with (i) the kinetics and/or extent of modification of the assay component being analyzed, and/or (ii) with the presence and/or activity of an enzyme (or modulator) that affects the modification. The nature of this step depends in part on whether the detectable response is simply detected or whether it is quantified. If the response is simply detected, it typically will be used to evaluate the presence of a component such as a substrate, product, and/or enzyme, or the presence of an activity such as an enzyme or modulator activity. In contrast, if the response is quantified, it typically will be used to evaluate the presence and/or quantity of a component such as a substrate, product, and/or enzyme, or the presence and/or activity of a component such as an enzyme or modulator.

The correlation generally may be performed using any suitable method, for example, by comparing the presence and/or magnitude of the response to another response (e.g., derived from a similar measurement of the same sample at a different time and/or another sample at any time) and/or a calibration standard (e.g., derived from a calibration curve, a calculation of an expected response, and/or a luminescent reference material). Such comparisons (and/or other procedures associated with the correlation) typically will be performed using a processor that is configured to receive and analyze a signal that corresponds to a detected response.

### IV. EXAMPLES

The following examples describe exemplary molecular modification assays involving lipids, lipid fragments, and/or lipid precursors, in accordance with aspects of the present teachings. These examples are included for illustration and are not intended to limit or define the entire scope of the present teachings.

### Example 1

### Exemplary Assays for Modification of Lipids

This example describes exemplary assays for modification of lipids; see FIGS. 3-5.

FIG. 3 shows a schematic view of exemplary lipid modification assays involving lipid phosphorylation and/or dephosphorylation. The assays may measure phosphorylation of a luminescent (“lum”) lipid with a kinase to add a phosphate group (“P”), and/or dephosphorylation of a luminescent phospholipid with a phosphatase. A binding partner (“BP”), such as a binding partner including a gallium ion, may be added to the reaction mixture. The binding partner may bind only to the phosphorylated form of the lipid, and not the nonphosphorylated form. Accordingly, addition of the binding partner may produce high fluorescence polarization (“high FP”) from the phosphorylated lipid, by binding to this form of the lipid. In contrast, the binding partner may not bind to the nonphosphorylated, luminescent lipid, resulting in low fluorescence polarization (“low FP”) measured from this form of the lipid. Therefore, conversion between these lipid forms may be assayed.

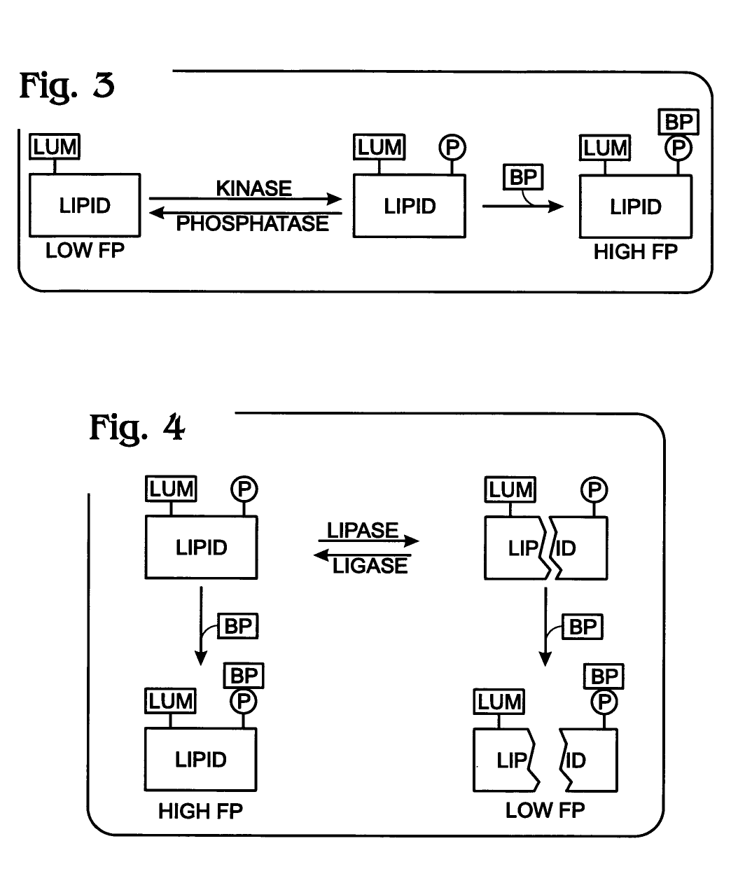

FIG. 4 shows a schematic view of exemplary lipid modification assays involving lipid cleavage and/or ligation. A luminescent (“lum”), phosphorylated (“P”) lipid may be cleaved by a lipase to form lipid fragments, or may be formed from lipid components or precursors by a ligase. When a binding partner is included in the reaction mixture, the binding partner may bind to each primary phosphate group. Linkage versus nonlinkage of the luminophore (“lum”) to the phosphate may determine an effect, if any, of the binding partner on fluorescence polarization. For example, on the left, the binding partner may bind to the lipid (and thus indirectly to the luminophore), thereby increasing the fluorescence polarization measured from the luminophore. By contrast, on the right, the binding partner may have no effect on the fluorescence polarization, because the luminophore and the phosphate group may be disposed on separate molecules after cleavage so that the binding partner does not bind indirectly to the luminophore.

In other embodiments, enzyme reaction may convert the phosphate group from a nonbinding configuration to a binding configuration (or vice versa). For example, the phosphate may be an internal phosphate that is exposed for binding by cleavage, or prevented from binding by ligation. Accordingly, in the presence of the binding partner, the cleaved/nonligated form of the lipid (or lipid precursor) may show higher fluorescence polarization than the noncleaved/ligated form.

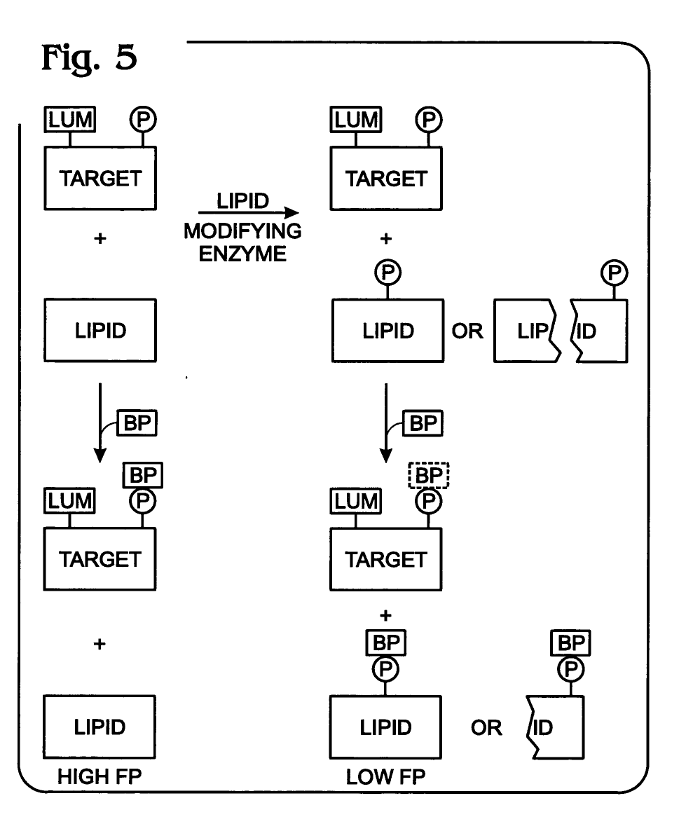

FIG. 5 shows a schematic view of exemplary lipid modification assays involving lipid cleavage and/or lipid phosphorylation/dephosphorylation. These assays may be performed as a competition with a “target” acting as a reporter. The reporter may be luminescent (“lum”) and may be configured to be bound by the binding partner (“BP”), such as by including a phosphate group (“P”) or other groups bound by the binding partner (for example, carboxyl or sulfonate groups, among others). In some examples, the target may include a plurality of negatively charged groups, and may be bound with lower affinity/specificity than the modified lipid.

The reaction may include a lipid. The lipid may be configured so that it does not bind to the binding partner until it is modified. Alternatively, the lipid may be configured so that it binds the binding partner until suitably modified. The lipid may be exposed to a lipid modifying enzyme, to assay the presence and/or activity of the enzyme. The enzyme may be configured, for example, to add a phosphate group to the lipid and/or to cleave the lipid, among others. In these examples, the enzyme may convert the lipid to a form that can be bound by the binding partner. Accordingly, the lipid modifying enzyme may increase the amount of lipid that can act as a competitor for a limiting amount of the binding partner. Therefore, the fluorescence polarization measured from the reporter may decrease as the lipid modifying enzyme activity increases.

### Example 2

### Exemplary Assays for the Modification of Lipid Precursors

This example describes exemplary assays for modification of a lipid precursor. In particular, this example describes assays for dephosphorylation of inositol monophosphate to inositol by the enzyme inositol monophosphatase.

Phosphoinositides may be synthesized by recycling the inositol released from phosphoinositides. The released inositol may be in a phosphorylated form, which may need to be dephosphorylated to provide nonmodified inositol as a precursor for phosphoinositide synthesis. For example, the enzyme inositol monophosphatase removes a phosphate from inositol monophosphate to produce myo-inositol. This enzyme may play a critical role in this recycling process, particularly in the mammalian brain, where it has been implicated as a significant factor in bipolar disorder (manic-depressive illness). This enzyme is inhibited noncompetitively by lithium, and may be a major target of lithium in the treatment of bipolar disorder. Accordingly, assays of inositol monophosphatase activity may be beneficial in clinical diagnosis or treatment, and/or may facilitate the identification of new drugs for treating bipolar disorder.

Assays for inositol monophosphate activity may be performed as follows. A substrate for the enzyme may be selected. Exemplary substrates may include a monophosphorylated inositol labeled with a luminescent moiety. The substrate may be combined with the enzyme (in purified form or in an extract), and incubated with the enzyme to allow the enzyme to catalyze dephosphorylation of the substrate. A binding partner, such as gallium ions, may be added to the reaction before, during, and/or after the enzyme reaction. Fluorescence polarization may be measured. This fluorescence polarization may decrease with increased enzyme activity, because the inositol product, in contrast to the substrate, may not bind to the binding partner.

### Example 3

### Exemplary Assays for Lipid Cleavage

This example describes exemplary assays for cleavage of lipids, particularly phosphoinositide lipids with the enzyme phospholipase C from mammals and/or bacteria.

Phosphoinositide (4,5) Bisphosphate, also known as Ptdlns(4,5)P2, is a substrate for the enzyme phosphoinositide-specific phospholipase C (PLC), particularly a mammalian form of the enzyme. Action of this enzyme results in formation of diacylglycerol (DAG) and inositol (3,4,5) trisphosphate (IP3). The activity of this enzyme may be associated with cell proliferation and invasion in human gliomas and thus may be a suitable target for high-throughput screens of enzyme modulators.

Assays for PLC activity may be performed as follows. A fluorescently tagged substrate may be selected. The fluorescent tag/label may be placed at any suitable position in the substrate, such as attached to a fatty acid chain, directly to the glycerol backbone, to phosphate, and/or to inositol. For example, the substrate may be BODIPY® FL C5, C6-phosphatidylinositol 4,5-diphosphate (available from Molecular Probes, Eugene, Oreg., as catalog number B22627), in which the fluorescent tag is attached to a fatty acid chain. The substrate may be combined with the phospholipase C enzyme (in purified form or in an extract), and incubated to permit the enzyme to cleave the substrate. A suitable detergent or other solubilizing agent also may be included. A binding partner, such as gallium ions, may be added, and then fluorescence polarization may be measured to determine the activity of the enzyme. Separation of the phosphorylated inositol head group from the fluorescent tag by the action of phospholipase C may prevent the binding partner from being associated with the fluorescent tag, thereby reducing the fluorescence polarization measured from this tag. Alternatively, the fluorescence polarization may increase with cleavage of the lipid.

In some embodiments, lipid cleavage assays may be performed without a binding partner. In particular, the fluorescence polarization of a fluorescently tagged lipid may change due to a change in the hydrophobicity of a tagged portion of the lipid produced by cleavage. For example, a tagged portion of the substrate may partition into (or out of) a micelle or vesicle, or may become insoluble (or soluble) due to separation of polar and hydrophobic regions of the lipid. Accordingly, in some embodiments, the binding partner may be omitted.

The activity of bacterial phosphoinositide phospholipase C also may be assayed. This enzyme cleaves phosphatidylinositol to 1,2 diacylglycerol and a myo-inositol cyclic 1,2-phosphate intermediate. Then, with slower kinetics, inositol 1-phosphate may be formed from the cyclic phosphate intermediate. (In contrast, the mammalian phosphoinositide phospholipase C may form a mixture of the cyclic and non-cyclic inositol products and diacylglycerol.)

Assays of enzyme activity may be performed by competition for a limiting amount of the binding partner. In particular, the assays may be based on a competition for the binding partner, between a fluorescent reporter and a product (or substrate) of the enzyme reaction. For example, the assays may include a fluorescent reporter that binds specifically to the binding partner, but is not modified by the enzyme being assayed. In the present illustration, the fluorescent target may be “FAM-CK1tide,” a 5-carboxyfluorescein-tagged peptide with four acidic residues. This peptide binds to a binding partner that includes gallium ions to produce a high fluorescence polarization. This binding and high fluorescence polarization can be competed with phosphate or phosphorylated substances, such as inositol 1-phosphate produced by phospholipase C.

Assays for bacterial phospholipase C activity may be performed as follows. Phospholipase C from Bacillus cereus may be contacted with phosphatidylinositol substrate at 37° C. for two hours. A concentrated competitor/binding partner solution may be prepared by mixing 20 μM FAM-CK1tide with 15 mM Ga(III)Cl in 0.1 N HCl. This concentrated solution then may be diluted in Buffer A (50 mM sodium acetate, 500 mM NaCl, pH 5.0) to produce a diluted solution having 150 nM peptide and 60 μM Ga(III). The diluted solution then may be added to the enzyme reaction. In some embodiments, an enzyme-dependent reduction in fluorescence polarization of about ΔmP=90 may be observed.

### Example 4

### Exemplary Assays for Phosphorylation of Lipids

This example describes exemplary assays for lipid kinase activity, particularly kinases that phosphorylate phosphoinositides.

Phosphoinositide 3-kinase (PI3K) places a phosphate group on the three position of the inositol moiety of phosphatitdyl inositol (Ptdlns), and may be an important target for drug development. For example, PI3K may play a key role in insulin signaling and may have reduced activity in tissues of subjects with type 2 diabetes. Furthermore, LY294002, a potent and selective PI3K inhibitor, may decrease growth of ovarian carcinoma and ascites formation in an athymic mouse xenogeneic transplant model of ovarian cancer. Accordingly, assays for PI3K may facilitate identification of new PI3K regulators.

PI3K assays may be performed using any suitable fluorescently labeled Ptdins as substrate. For example, the Ptdlns may be labeled with BODIPY® FL, TMR or NBS, and thus may be obtained from Molecular Probes, Eugene, Oreg. and/or Echelon, Salt Lake City, Utah. Fatty acid chains of any suitable length may be included in the substrate, such as chains of five, six, and/or sixteen carbons, among others. Detergents and/or lipids may be included in enzyme reactions and/or binding reactions to produce micelles and/or vesicles and/or to facilitate solubilizing the substrate (and/or product). Action of PI3K phosphorylates the fluorescent Ptdins, which may allow it to bind to a binding partner, resulting in increased fluorescence polarization.

Any suitable PI3K enzyme may be used in the assays. For example, the PI3K may be in crude or purified extracts, and/or may be obtained commercially. In particular, an isoform, p110y, may be obtained from Sigma or Alexa, and may be used without activators or regulatory subunits. Calibration curves may be generated in binding reactions with varying ratios of nonphosphorylated/phosphorylated substrate (“substrate/calibrator”), such as varying ratios of C6 BODIPY® PtdIns/PtdIns(3)P, to facilitate selection of assay conditions.

### Example 5

### Exemplary Assays for Dephosphorylation of Lipids

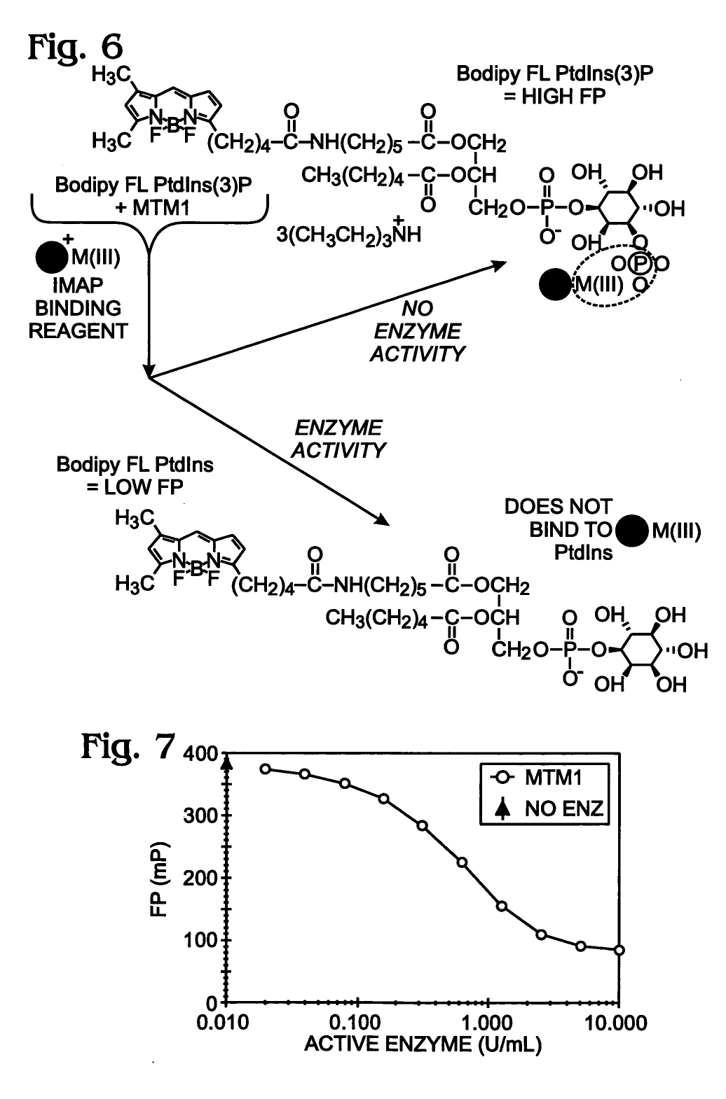

This example describes exemplary assays for lipid phosphatase activity, particularly acitvity of phosphatases that dephosphorylate phosphoinositides; see FIGS. 6 and 7.

MTM (myotubularin) and PTEN are phosphoinositide phosphatases. Each of these phosphatases may be capable of removing the phosphate from the number three position of inositol in phosphoinositides and may be specific for this phosphate position. These proteins may have important roles in signaling. For example, PTEN may be a tumor suppressor protein, and may be disregulated in oncogenesis such as in prostate and ovarian forms of cancer. Each of these enzymes may be available in a purified form from Upstate, Waltham, Mass.

FIG. 6 shows a schematic representation of a strategy for measuring the activity of lipid phosphatases, such as MTM1 or PTEN. An exemplary phosphoinositide substrate (BODIPY® FL C5, C6-phosphatidylinositol 3-phosphate (BODIPY® FL C5, C6-Ptdins(3)P) from Molecular Probes, Eugene, Oreg., may be selected for use in the assays. The substrate may bind to a binding partner (M^{III}) in its nonmodified form (“no enzyme activity”), to increase the fluorescence polarization measured from the substrate (“high FP”). However, the substrate may be dephosphorylated by a phosphatase, such as MTM1 (“enzyme activity”), to produce a product that doesn't bind to the binding partner and therefore produces lower fluorescence polarization (“low FP”).

Assays may be performed as follows. Exemplary enzyme reactions may be performed in a reaction buffer including 10 mM Tris-HCl, 10 mM MgCl₂, 0.1% BSA, 0.05% NaN₃, 1 mM DTT, pH 7.2, with 100 nM BODIPY® FL C5, C6-Ptdlns(3)P for sixty minutes. A binding solution with 50 mM sodium acetate, 500 mM NaCl, 75 μM Ga(III), pH 5.0, then may be added and the mixture incubated for thirty minutes. Fluorescence polarization may be measured. A detergent may be included in the reaction buffer and/or the binding solution. For example, 0.1% Brij may be included in the binding solution, or 0.1% Triton X-100 or Brij may be included in the reaction buffer.

FIG. 7 shows a graph of assay data with myotubularin 1. Fluorescence polarization measurements (mP) were plotted against the concentration of myotubularin 1 (MTM1) enzyme in the assays. Each point represents the average of four replicates. In the present illustration, the enzyme reaction results in dephosphorylation of the inositol head group and loss of binding to Ga(III), thereby producing reduced fluorescence polarization with increased enzyme activity. In other embodiments, PTEN may be used as enzyme and/or TMR C6 Ptdins(3)P from Echelon, Salt Lake City, Utah, may be used as substrate. Alternatively, or in addition, longer (and/or shorter) fatty acid chains may be used on the substrate.

### Example 6

### Selected Embodiments I

This example, set forth in the attached appendix, describes further aspects and embodiments of the invention.

### Example 7

### Selected Embodiments II

This example describes selected aspects and embodiments of the invention, as a series of ordered paragraphs.

1. A method of detecting modification of a lipid or lipid precursor in a sample, comprising: (A) contacting the sample with a binding partner that binds specifically to a modified form or a nonmodified form of the lipid or lipid precursor, but not both, the binding partner including a metal required for specific binding to the one form; and (B) detecting a response indicative of the extent of binding between the binding partner and the one form.

2. The method of paragraph 1, wherein the detected response is indicative of a decrease in the nonmodified form of the lipid.

3. The method of paragraph 1 or 2, wherein the detected response is indicative of the presence of the nonmodified form of the lipid.

4. The method of any preceding paragraph, wherein the detected response includes a luminescence polarization response.

5. The method of any preceding paragraph, wherein the detected response includes a change in luminescence resonance energy transfer.

6. The method of any preceding paragraph, wherein the detected response includes at least one of a change in the intensity, polarization, energy transfer, lifetime, and excitation or emission wavelength distribution of a detected luminescence.

7. The method of any preceding paragraph, wherein the modified and nonmodified forms are related by an enzymatic modification.

8. The method of paragraph 7, the enzymatic modification being mediated by an enzyme, further comprising adding a candidate modulator of activity of the enzyme to the sample, prior to the step of detecting a response.

9. The method of paragraph 7, wherein the enzymatic modification is cleavage.

10. The method of paragraph 9, wherein the enzymatic modification cleaves the lipid or the lipid precursor between a phosphate moiety and a luminescent moiety.

11. The method of paragraph 7, wherein the enzymatic modification is phosphorylation.

12. The method of any preceding paragraph, wherein the extent of binding between the binding partner and the one form is related to one or more functional groups of the one form.

13. The method of paragraph 12, wherein the functional group is ionic at physiological pH.

14. The method of paragraph 12, wherein the functional group is selected from phosphate, sulfonate, and carboxylate.

15. The method of any preceding paragraph, wherein the binding partner includes a cationic metal ion.

16. The method of paragraph 15, wherein the binding partner includes a gallium ion.

17. The method of paragraph 15, wherein the binding partner includes a particle or macromolecule associated with the metal ion.

18. The method of any of paragraphs 1-16, wherein the method is performed with a lipid, and wherein the lipid is a phosphoinositide.

19. The method of any of paragraphs 1-16, wherein the method if performed with a lipid precursor, and wherein the lipid precursor includes inositol.

20. The method of any preceding paragraph, wherein the step of contacting also contacts the binding partner with a luminescent reporter to which the binding partner can bind, and wherein the step of detecting includes a step of measuring competition, if any, between the one form and the luminescent reporter for binding to the binding partner.

21. The method of any preceding paragraph, wherein the step of contacting is performed in the presence of a detergent that increases the response relative to no detergent.

22. A kit configured to perform the method of any one of paragraphs 1 to 21.

23. The kit of paragraph 22, comprising a binding partner.

24. The kit of paragraph 23, wherein the binding partner is disposed in a liquid.

25. The kit of paragraph 22, comprising an enzyme substrate that is luminescent.

26. The kit of paragraph 22, further comprising one or more calibration standards.

The disclosure set forth herein may encompass one or more distinct inventions, each with independent utility. Although these inventions have been disclosed in their preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of detecting modification of a lipid or lipid precursor in a sample, comprising:
contacting the sample with a binding partner that binds specifically to a modified form or a nonmodified form of the lipid or lipid precursor, but not both, the binding partner including a metal required for specific binding to the one form; and
detecting a response indicative of the extent of binding between the binding partner and the one form.

2. The method of claim 1, wherein the detected response is indicative of a decrease in the nonmodified form of the lipid.

3. The method of claim 1, wherein the detected response is indicative of the presence of the nonmodified form of the lipid.

4. The method of claim 1, wherein the detected response includes a luminescence polarization response.

5. The method of claim 1, wherein the detected response includes a luminescence resonance energy transfer response.

6. The method of claim 1, wherein the detected response includes at least one of a change in the intensity, polarization, energy transfer, lifetime, and excitation or emission wavelength distribution of a detected luminescence.

7. The method of claim 1, wherein the modified and nonmodified forms are related by an enzymatic modification.

8. The method of claim 7, the enzymatic modification being mediated by an enzyme, further comprising adding a candidate modulator of activity of the enzyme to the sample, prior to the step of detecting a response.

9. The method of claim 7, wherein the enzymatic modification is cleavage.

10. The method of claim 9, wherein the enzymatic modification cleaves the lipid or the lipid precursor between a phosphate moiety and a luminescent moiety.

11. The method of claim 7, wherein the enzymatic modification is phosphorylation.

12. The method of claim 1, wherein the extent of binding between the binding partner and the one form is related to one or more functional groups of the one form.

13. The method of claim 12, wherein the functional group is ionic at physiological pH.

14. The method of claim 12, wherein the functional group is selected from phosphate, sulfonate, and carboxylate.

15. The method of claim 1, wherein the binding partner includes a cationic metal ion.

16. The method of claim 15, wherein the binding partner includes a gallium ion.

17. The method of claim 15, wherein the binding partner includes a particle or macromolecule associated with the metal ion.

18. The method of claim 1, wherein the method is performed with a lipid, and wherein the lipid is a phosphoinositide.

19. The method of claim 1, wherein the method if performed with a lipid precursor, and wherein the lipid precursor includes inositol.

20. The method of claim 1, wherein the step of contacting also contacts the binding partner with a luminescent reporter to which the binding partner can bind, and wherein the step of detecting includes a step of measuring competition, if any, between the one form and the luminescent reporter for binding to the binding partner.

21. The method of claim 1, wherein the step of contacting is performed in the presence of a detergent that increases the response relative to no detergent.

22. A kit configured to perform the method of claim 1.
